(19) United States
(12) Patent Application Publication
(10) Pub. No.: US 2019/0142483 A1
(43) Pub. Date: May 16, 2019

(54) OSTEOSYNTHESIS PLATE AND OSTEOSYNTHESIS KIT COMPRISING SAID PLATE

(71) Applicant: SIM SOLUZIONI INNOVATIVE MEDICALI SAGL, CHIASSO-TICINO (CH)

(72) Inventor: Nilli Del Medico, Orbassano (IT)

(21) Appl. No.: 15/769,822

(22) PCT Filed: Oct. 22, 2015
(86) PCT No.: PCT/IB2015/058160

§ 371 Date: Apr. 20, 2018

(51) Int. Cl. A61B 17/80

(52) U.S. Cl. CPC A61B 17/8033; A61B 17/74; A61B 17/8004

(57) ABSTRACT

An osteosynthesis plate for being used either in a static locking mode or in a dynamic locking mode is provided. The osteosynthesis plate comprises a flat, elongated body in which there are provided two or more slots intended to receive corresponding screws for securing the plate to a fractured bone, the slots and the screws being mutually cooperating for preventing the risk of misalignments when the plate is used in a dynamic locking mode. Retaining elements for the screws are provided in the slots, the elements preventing, either totally or selectively, a displacement of the screws within the slots. An osteosynthesis kit allowing to use the aforesaid plate as static osteosynthesis plate or as dynamic osteosynthesis plate, as needed, is also provided.

### TECHNICAL FIELD

The present invention relates to an osteosynthesis plate.

The present invention further relates to an osteosynthesis kit comprising the aforesaid osteosynthesis plate.

### PRIOR ART

Osteosynthesis plates for treating fractured bones, especially for fractures in long bones such as the femur or the tibia, are known.

Said plates usually have an elongated shape and have a plurality of through-holes for the passage of screws or similar fasteners allowing to secure the plate to the fractured bone, particularly to the diaphysis and/or epiphysis of the fractured bone.

Osteosynthesis plates can be divided into two broad categories: osteosynthesis plates with static locking and osteosynthesis plates with dynamic locking.

In principle, the osteosynthesis process is divided into two steps:
in a first step, immediately after the fracture has been reduced and the osteosynthesis plate has been applied, the fractured bone is not subjected to loads (especially loads along the direction parallel to the bone axis) in order to promote proper formation of the bone callus;
in a second step, when the fracture is already partially consolidated, the bone is then subjected to increasingly higher loads for accelerating healing thereof.

Depending on the specific circumstances—and especially on the kind and position of the fracture, on the general conditions of the patient and so on—it may be preferable to use a osteosynthesis plate with static locking (or plate for static osteosynthesis), exerting a rigid compression constant over time, or an osteosynthesis plate with dynamic locking (or plate for dynamic osteosynthesis), in which the exerted compression varies according to the level of progression of the osteosynthesis process, i.e. according to the time elapsed from the reduction of the fracture and/or to the entity of the load applied to the bone to which the plate is attached.

Several different types of static osteosynthesis plates as well as several types of dynamic osteosynthesis plates are known.

The main object of the invention is to provide an osteosynthesis plate suitable for being used, in a profitable way and according to the specific needs, either as static osteosynthesis plate or as dynamic osteosynthesis plate.

Another object of the invention is to provide an osteosynthesis plate which, when used as a dynamic osteosynthesis plate, allows to keep the plate itself as well as its components in proper position.

Another object of the present invention is to provide an osteosynthesis plate which, when used as a dynamic osteosynthesis plate, allows to obtain gradual varying of compression without abrupt changes.

A further object of the present invention is to provide an osteosynthesis kit allowing to use the aforementioned plate as plate for static osteosynthesis or as plate for dynamic osteosynthesis, as needed.

A further object of the present invention is to provide an osteosynthesis kit enabling the orthopaedic surgeon to decide whether to use the aforementioned plate as plate for static osteosynthesis or as plate for dynamic osteosynthesis at any time, even during surgery.

These and other objects are achieved by the osteosynthesis plate and osteosynthesis kit as claimed in the appended claims.

### SUMMARY OF THE INVENTION

The osteosynthesis plate according to the invention is suitable for being selectively used as plate for static osteosynthesis or as plate for dynamic osteosynthesis.

The osteosynthesis plate according to the invention comprises a flat, elongated body in which one or more slots are provided arranged along the longitudinal axis of said body and substantially aligned to said longitudinal axis, said slots being adapted to receive corresponding screws for securing said plate to a fractured bone.

Owing to the fact that the slots in the osteosynthesis plate according to the invention are delimited by a pair of mutually parallel walls facing each other and that the screws that are received in said slots have an unthreaded, cylindrical surface portion which cooperates with said parallel, facing walls, when said plate is used as plate for dynamic osteosynthesis the screws that are received in said slots are guided along a rectilinear path without possibility of any misalignment or deviation.

Due to the fact that, according to the invention, retaining means for said screws are provided in said slots, sliding of said screws relative to the plate body in the direction of the longitudinal axis of said plate within said slots can be totally or selectively prevented. According to a first preferred embodiment of the invention, said retaining means are made of a substantially rigid material, for instance metal, whereby sliding of the screw inside its corresponding slot can be prevented for the entire time of use of the osteosynthesis plate.

According to a second preferred embodiment of the invention, said retaining means are made of a bioreabsorbable material, for instance a bioreabsorbable polymer, whereby a sliding of the screw within its corresponding slot is allowed, said sliding becoming increasingly greater over time, as the retaining means are absorbed by the patient's organism.

According to a possible embodiment of the invention, said retaining means are designed as a shim having such size and shape that it can be inserted into a slot of said osteosynthesis plate and prevent any sliding of the corresponding screw within said slot.

According to a possible alternative embodiment of the invention, said retaining means are designed as a peg that can be inserted into corresponding seats formed on opposite walls of a slot of said osteosynthesis plate so as to extend in a transverse direction within said slot and prevent any sliding of the corresponding screw within said slot. According to a particularly advantageous variant of this embodiment, the opposite walls of said slots provide a plurality of corresponding seats that are mutually aligned and spaced apart, whereby the position of said transverse peg can be chosen among a plurality of options.

Advantageously, the present invention further relates to an osteosynthesis kit comprising an osteosynthesis plate according to the invention, a plurality of retaining means made of a substantially rigid material and/or a plurality of retaining means made of a bioreabasorbable material.

### BRIEF DESCRIPTION OF THE DRAWINGS

Further features and advantages of the invention will become apparent form the following detailed description of some preferred embodiments of the invention, which are given by way of non-limiting example with reference to the annexed drawings, in which:

FIG. 1 is a schematic view showing an osteosynthesis plate according to the invention;

FIG. 1a is a schematic sectional view of the osteosynthesis plate of FIG. 1 taken along the line A-A

FIG. 2a is an enlarged scale view of a detail of the osteosynthesis plate of FIG. 1 according to a first preferred embodiment of the invention;

FIG. 2b is an enlarged scale view of a detail of the osteosynthesis plate of FIG. 1 according to a second preferred embodiment of the invention;

FIG. 2c is an enlarged scale view of a detail of the osteosynthesis plate of FIG. 1 according to a third preferred embodiment of the invention;

FIG. 2d is an enlarged scale view of a detail of the osteosynthesis plate of FIG. 1 according to a fourth preferred embodiment of the invention;

FIG. 2e is an enlarged scale view of a detail of the osteosynthesis plate of FIG. 1 according to a fourth preferred embodiment of the invention.

### DESCRIPTION OF SOME PREFERRED EMBODIMENTS OF THE INVENTION

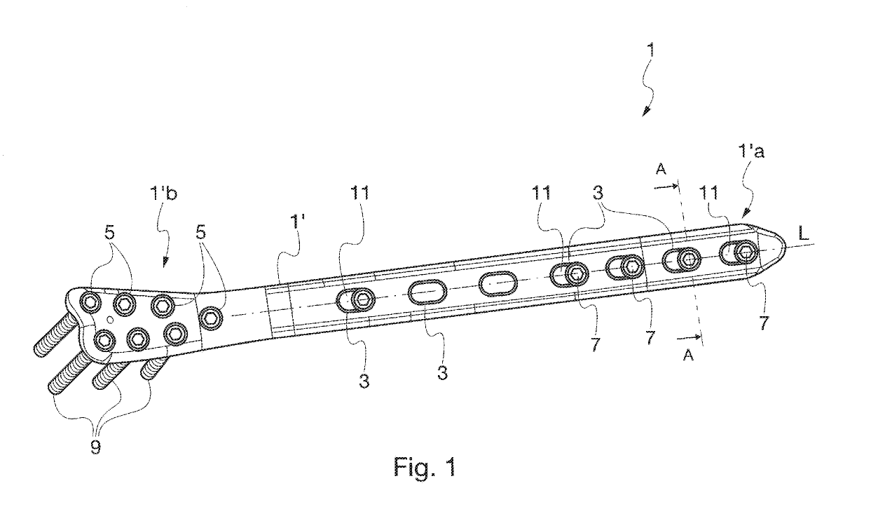

Referring at first to FIG. 1, this illustrates an example of an osteosynthesis plate according to the invention, designated as a whole by reference numeral 1.

In the shown example, the osteosynthesis plate 1 is a plate suitable for treating fractures of long bones such as the femur, and especially for treating diaphyseal fractures of long bones.

Said osteosynthesis plate 1 comprises a flat, elongated body 1′ extending along a longitudinal axis L thereof and comprising a first portion 1′a adapted to be secured to the middle portion or diaphysis of a fractured bone, and a second portion 1′b adapted to be secured to the head or epiphysis of the fractured bone.

In a manner known per se, cortical screws 7 are provided for securing the first portion 1′a of the body 1′ of the osteosynthesis plate 1 to the diaphysis of the fractured bone and cephalic screws 9 are provided for securing the second portion 1′b of the body 1′ of the osteosynthesis plate 1 to the epiphysis of the fractured bone.

According to the invention, the body 1′ of the osteosynthesis plate 1 comprises one or more slots 3 arranged along the longitudinal axis L of said plate 1 and substantially aligned with said longitudinal axis L for receiving corresponding securing screws.

More particularly, in the example of FIG. 1, slots 3 are provided in the first portion 1′ a of said body 1′ for receiving corresponding cortical screws 7 for securing the plate 1 to the diaphysis of a fractured bone.

In the example of FIG. 1, substantially round holes 5 are provided in the second portion 1′b of said body 1′ for receiving corresponding cephalic screws 9 for securing the plate 1 to the epiphysis of a fractured bone.

Obviously, it is possible to imagine that the body 1′ of the osteosynthesis plate 1 comprises slots also in the second portion 1′b of said body 1′ for receiving corresponding cephalic screws 9 in said slots.

Furthermore, depending on the specific requirements, it is possible to imagine that the body 1′ of the osteosynthesis plate 1 comprises slots only in the second portion 1′b of said body 1′ for receiving corresponding cephalic screws 9 in said slots, the cortical screws 7 being received in substantially round holes in the first portion 1′a of said body 1′.

According to the invention—as better visible in FIG. 1a—the slots 3 are delimited by a pair of walls 3c, 3d that are parallel (to each other and to the longitudinal axis of the plate 1) and facing each other, and joined to each other at opposite ends by means of curvilinear joining portions. Correspondingly, the cortical screws 7 received in said slots 3 comprise a lateral wall portion 7a having an unthreaded, cylindrical shape which—when one of said screws 7 is inserted in a corresponding slot 3—cooperates with said parallel and facing opposite walls 3c, 3d of said slot. In other words, said cortical screws 7 comprise a lateral wall portion 7a having a cylindrical shape and a diameter substantially equal to the distance between the parallel, mutually facing walls 3c, 3d of the slot 3.

Advantageously, by virtue of the positive mechanical engagement and cooperation between the lateral walls 3c, 3d of the slot 3 and the cylindrical lateral wall portion 7a of the screw 7 received therein, said slot 3 acts as a rail for said screw 7, which can slide along the slot as in a guiding rail, but it is prevented from effecting any rotational movements relative to the plane on which the body 1′ of the plate 1 lies, thus avoiding any risks of misalignments and deviations and guaranteeing proper positioning of the plate 1 and its components during the entire osteosynthesis process.

The cylindrical lateral wall portion 7a of the screw 7 may be a lateral wall portion of the screw shaft. However, according to a preferred embodiment of the invention, the screws 7 are screws with cylindrical head and said cylindrical lateral wall portion 7a of the screw 7 is the lateral surface of the head of the screw 7 or part thereof.

Still according to the invention, the osteosynthesis plate 1 may comprise, for each slot 3, retaining means 11 adapted to prevent and/or limit and/or control the displacement of the corresponding cortical screw 7 within said slot, i.e. to prevent and/or limit and/or control the displacement of the corresponding cortical screw 7 relative to the body 1′ of the plate 1 in the direction of the longitudinal axis L of said plate 1.

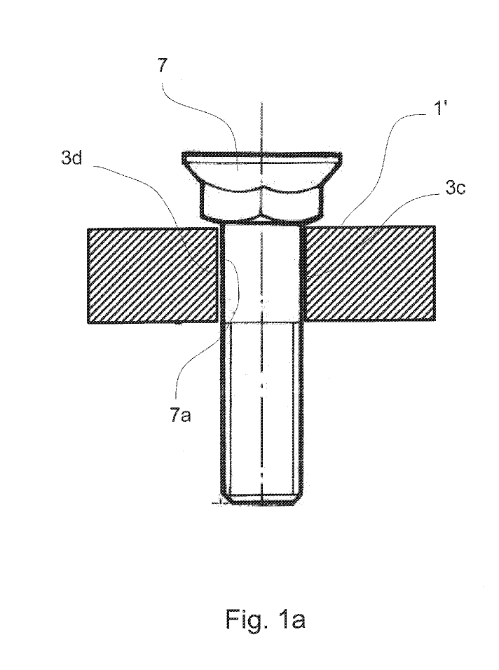

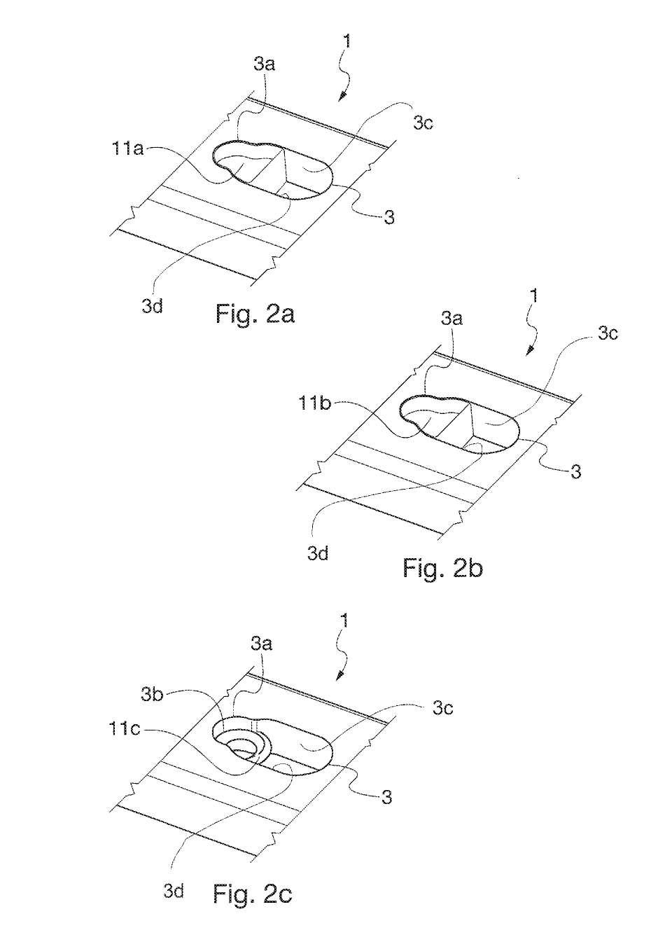

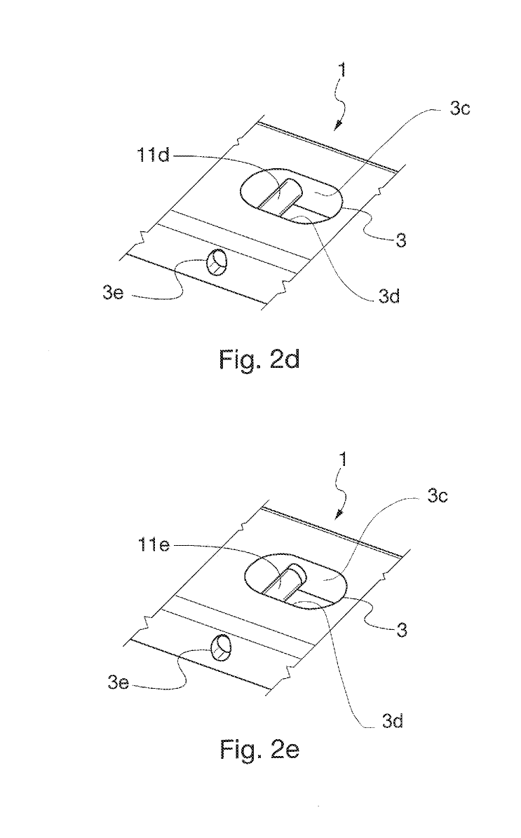

For the sake of clarity, said retaining means 11 are not shown in FIG. 1 and will be described below with reference to FIGS. 2a-2d.

FIGS. 2a-2e illustrate some preferred embodiments of the retaining means 11 of the osteosynthesis plate 1 according to the invention.

In the first preferred embodiment of the invention, shown in FIG. 2a, the retaining means are designed as a shim 11a made of a substantially rigid material and having such shape and size that it can be inserted into a slot 3 of said osteosynthesis plate 1 and prevent any sliding of the corresponding screw within said slot 3.

To this aim, the slot 3 may comprise a shaped end portion 3a for facilitating insertion of the shim 11a into the slot.

In this embodiment, the shim 11a can be made of metal, for instance of a metal suitable for surgical applications, such as titanium.

The use of retaining means according to said first embodiment of the invention, made of a substantially rigid material, allows a static locking of the osteosynthesis plate, because the cortical screws 7 inserted in the slots 3 cannot slide relative to the body of the osteosynthesis plate 1 for the entire period of time for which the plate 1 is attached to the fractured bone.

It is optionally possible to envisage a variant of the first embodiment of the invention illustrated in FIG. 2a, in which the shim 11a is made of a substantially rigid material, but it has such shape and size that it leaves, within the slot 3, a residual free space in which the corresponding screw can freely move.

In this variant, some possibility of sliding of the screw 7 relative to the slot 3 is allowed and the amount of such sliding is fixed for the entire period of time for which the plate 1 is attached to the fractured bone.

In the second preferred embodiment of the invention, shown in FIG. 2b, the retaining means are designed as a shim 11b made of a bioreabsorbable material and having such shape and size that it can be inserted into a slot 3 of said osteosynthesis plate 1 and prevent—upon application thereof—any sliding of the corresponding screw within said slot 3.

In this case, too, the slot 3 may comprise a shaped end portion 3a for facilitating both insertion and retaining of the shim 11b in the slot.

In this embodiment, the shim 11b can be made of a bioreabsorbable polymer, such as for instance polylactic acid.

The use of retaining means according to said first embodiment of the invention, made of a bioreabsorbable material, allows a dynamic locking of the osteosynthesis plate: at first the cortical screws 7 inserted in the slots 3 cannot slide relative to the body of the osteosynthesis plate 1; subsequently, as the shim 11b is reabsorbed, the free space within the slot 3 increases progressively, thus allowing a displacement of the screw 7 within the slot 3, such displacement becoming increasingly greater over time.

In the third preferred embodiment of the invention, shown in FIG. 2c, the retaining means are designed as a ring 11c made of a substantially rigid material and having such shape and size that it can be inserted into a slot 3 of said osteosynthesis plate 1 and prevent any sliding of the corresponding screw within said slot 3.

To this aim, the slot 3 may comprise a shaped end portion 3a and a groove 3b at said shaped end portion 3a for allowing insertion and retainment of the ring 11c in the slot.

As in the first embodiment, in this embodiment, too, the ring 11c can be made of metal, for instance titanium.

The use of retaining means according to said third embodiment of the invention, made of a substantially rigid material, allows a static locking of the osteosynthesis plate.

In the fourth preferred embodiment of the invention, shown in FIG. 2d, the retaining means are designed as a peg 11d made of a substantially rigid material and inserted transversely through a slot 3 of the plate 1 in such a position as to be able to prevent any sliding of the corresponding screw within said slot 3.

To this aim, the slot 3 comprises a pair of aligned holes 3c which are provided on the opposite flanks the body of the plate 1 and open on the respective opposite walls 3c, 3d of the slot 3, at the desired position relative to the slot 3, and through which the transverse peg 11d can be inserted.

As in the first embodiment, in this embodiment, too, the transverse peg 11d can be made of metal, for instance titanium.

The use of retaining means according to said fourth embodiment of the invention, made of a substantially rigid material, allows a static locking of the osteosynthesis plate.

It is optionally possible to envisage a variant of the fourth embodiment of the invention illustrated in FIG. 2d, in which the opposite flanks of the body of the plate 1 are provided, at each slot 3, with a plurality of corresponding aligned, spaced apart holes. In this way, the transverse peg 11d can be inserted in a position chosen among a plurality of different options.

In the fifth preferred embodiment of the invention, shown in FIG. 2e, the retaining means are designed as a peg 11e made of a bioreabsorbable material and inserted transversely through a slot 3 of the plate 1 in such a position as to be able to prevent—upon application thereof—any sliding of the corresponding screw within said slot 3.

To this aim, the slot 3 comprises a pair of aligned holes 3e, which are provided on the opposite flanks of the body of the plate 1 and open on the respective opposite walls 3c, 3d of the slot 3, at the desired position relative to the slot 3, and through which the transverse peg 11e can be inserted.

As in the second embodiment, in this embodiment, too, the transverse peg 11e can be made of a bioreabsorbable polymer, for instance of polylactic acid.

The use of retaining means according to said fifth embodiment of the invention, made of a bioreabsorbable material, allows a dynamic locking of the osteosynthesis plate.

In this case, too, it is optionally possible to envisage a variant in which the opposite flanks of the body of the plate 1 are provided, at each slot 3, with a plurality of corresponding aligned, spaced apart holes, whereby it is possible to choose in which pair of holes the transverse peg 11e is to be inserted, thus determining a different position of said peg.

It is to be noted that, unlike the second embodiment, in which the gradual absorption of the shim 11b made of reabsorbable material causes a progressive increase of the free space within the slot 3 in which the corresponding screw can slide, in the fifth embodiment illustrated in FIG. 2e, there will be a shift from a situation in which the transverse peg 11e has not been reabsorbed and the screw is locked in position to a situation in which the transverse peg 11e has been reabsorbed and the screw can slide freely over the entire extension of the slot 3.

It is clear from the above description that the osteosynthesis plate according to the invention achieves the objects set forth above by allowing several degrees of freedom and, particularly, it is suitable for being used either in a static locking mode or in a dynamic locking mode.

It will be further evident to those skilled in the art that it will advantageously be possible to envisage to provide an osteosynthesis kit to be made available to the orthopaedic surgeon, said kit containing:
an osteosynthesis plate according to claim 1;
a first plurality of retaining means 11a;11c;11d made of a substantially rigid material; and/or
a second plurality of retaining means 11b;11e made of a bioreabsorbable material.

In this way, the orthopaedic surgeon, depending on the specific situation, will be able to choose the most suitable retaining means among those available in the osteosynthesis kit.

If the orthopaedic surgeon chooses to use the osteosynthesis plate according to the invention as dynamic locking plate, the positive mechanical engagement and cooperation between the osteosynthesis screws and the slots receiving them will guarantee that the risk of misalignments is prevented.

Of course the embodiments illustrated here have been given merely by way of example and several modifications and variants are possible without departing from the scope of the protection as defined by the appended claims.

More particularly, as anticipated above, the invention may be applied both to the positioning of cortical screws and to the locking of cephalic screws as well as to the locking of both kinds of screws.

In addition, within a same application, retaining means of different size, shape and kind may be used in different slots if required or advisable in view of the circumstances.

1: An osteosynthesis plate comprising a flat, elongated body extending along a longitudinal axis thereof and securing screws for securing said body of said plate to a fractured bone, wherein said body of said plate is provided with one or more slots intended to receive respective securing screws and arranged along said longitudinal axis of said plate, substantially in line with said longitudinal axis, wherein said slots are delimited by a pair of walls, said walls being parallel to each other and to said longitudinal axis, being facing each other and being joined to each other at opposite ends by curvilinear joining portions and wherein one or more of said securing screws received in said slots comprise a lateral wall portion having a cylindrical, unthreaded form with a diameter substantially equal to the distance between said parallel, mutually facing walls of the corresponding slots receiving said screws.

2: The osteosynthesis plate according to claim 1, wherein, one or more of said securing screws are screws with cylindrical head and said lateral wall portion having an unthreaded, cylindrical form is the lateral surface of the head of said screws or part thereof.

3: The osteosynthesis plate according to claim 1, wherein retaining elements are provided in each of said one or more slots, said retaining elements being suitable for preventing, either totally or selectively, a displacement of the corresponding securing screw relative to said body of said plate in the direction of the longitudinal axis of said plate.

4: The osteosynthesis plate according to claim 3, wherein said retaining elements are made of a substantially rigid material, preferably of metal.

5: The osteosynthesis plate according to claim 4, wherein said retaining elements are designed as a shim fitted into said slot.

6: The osteosynthesis plate according to claim 4, wherein said retaining elements are designed as a ring fitted into a groove provided in said slot.

7: The osteosynthesis plate according to claim 4, wherein at least a pair of aligned holes are provided in the opposite flanks of said body of said plate and wherein said retaining elements are designed as a peg transversely inserted into said slot through said holes.

8: The osteosynthesis plate according to claim 3, wherein said retaining elements are made of a bioreabsorbable material.

9: The osteosynthesis plate according to claim 8, wherein said retaining elements are designed as a shim fitted into said slot.

10: The osteosynthesis plate according to claim 8, wherein at least one pair of aligned holes are provided in the opposite flanks of said body of said plate and wherein said retaining elements are designed as a peg transversely inserted into said slot through said holes.

11: The osteosynthesis plate according to claim 10, wherein said opposite flanks of said body of said plate are respectively provided, at each of said slots, with a plurality of aligned, spaced holes.

12: An osteosynthesis kit comprising:
an osteosynthesis plate comprising a flat, elongated body extending along a longitudinal axis thereof and securing screws for securing said body of said plate to a fractured bone, wherein said body of said plate is provided with one or more slots intended to receive respective securing screws and arranged along said longitudinal axis of said plate, substantially in line with said longitudinal axis, wherein said slots are delimited by a pair of walls, said walls being parallel to each other and to said longitudinal axis, being facing each other and being joined to each other at opposite ends by curvilinear joining portions and wherein one or more of said securing screws received in said slots comprise a lateral wall portion having a cylindrical, unthreaded form with a diameter substantially equal to the distance between said parallel, mutually facing walls of the corresponding slots receiving said screws;
a plurality of retaining elements made of a substantially rigid material.

13: The osteosynthesis plate according to claim 8, wherein said retaining elements are made of a bioreabsorbable polymer.

14: The osteosynthesis plate according to claim 7, wherein said opposite flanks of said body of said plate are respectively provided, at each of said slots, with a plurality of aligned, spaced holes.

15: An osteosynthesis kit comprising:
an osteosynthesis plate comprising a flat, elongated body extending along a longitudinal axis thereof and securing screws for securing said body of said plate to a fractured bone, wherein said body of said plate is provided with one or more slots intended to receive respective securing screws and arranged along said longitudinal axis of said plate, substantially in line with said longitudinal axis, wherein said slots are delimited by a pair of walls, said walls being parallel to each other and to said longitudinal axis, being facing each other and being joined to each other at opposite ends by curvilinear joining portions and wherein one or more of said securing screws received in said slots comprise a lateral wall portion having a cylindrical, unthreaded form with a diameter substantially equal to the distance between said parallel, mutually facing walls of the corresponding slots receiving said screws;
a plurality of retaining elements made of a bioreabsorbable material.
